(19) United States
(12) Patent Application Publication
(10) Pub. No.: US 2026/0264962 A1
(43) Pub. Date: Sep. 10, 2026

(54) A PACKAGING SYSTEM WITH INTEGRATED STERILISATION

(71) Applicant: Technological University of the Shannon: Midlands Midwest, Athlone (IE)

(72) Inventors: Luke Geever, Athlone (IE); Maya Frost, Athlone (IE); Laura Grehan, Athlone (IE); Cathal McGrath, Athlone (IE); Crevan O’Donnell, Athlone (IE); Seán Egan, Galway (IE); Lyndsey McMillan, Galway (IE); Gavin Keane, Athlone (IE); Alan Kelly, Galway (IE); Joe Holland, Galway (IE)

(21) Appl. No.: 19/165,712

(22) PCT Filed: Mar. 21, 2024
(86) PCT No.: PCT/EP2024/057575

§ 371 Date: Sep. 16, 2025

(30) Foreign Application Priority Data
Mar. 21, 2023 (GB) 2304106.4

(51) Int. Cl. B65D 81/28; A61L 2/20; A61L 103/00; B65D 81/32

(52) U.S. Cl. CPC B65D 81/28; A61L 2/20; B65D 81/3294; A61L 2103/23; B65D 2203/12

(57) ABSTRACT

The present invention provides a packaging system with integrated sterilisation, and in particular a packaging system for medical devices which is adapted to sterilise the packaged medical device following sealing within the packaging system in a safe and convenient manner, the packaging system including a rigid container defining a main compartment and an integrally formed auxiliary compartment arranged to receive a module operable through external activation to release a gaseous sterilising agent into the main compartment to sterilise a medical device located therein.

### ACKNOWLEDGEMENT

The projects leading to this application received funding from Enterprise Ireland.

### FIELD OF INVENTION

The present invention relates to a packaging system with integrated sterilisation, and in particular a packaging system for medical devices, pharmaceuticals, food products or the like which is adapted to decontaminate or sterilise the packaged item following sealing within the packaging system.

### BACKGROUND OF THE INVENTION

Sterilisation technologies for items such as medical devices have remained essentially unchanged for several decades. The complete sterilisation process can be both lengthy and expensive, increasing final product lead times and reducing cost effectiveness. There is a long felt need for the ability to sterilise products in-house in a reliable, fast and low-cost manner. Recently the existing methods of sterilisation have become problematic, with the seepage of carcinogenic materials into the localities where the sterilisation plants are located, prompting the closures of plants by the FDA (MedTech Dive, April 2019).

Known methods for decontamination or sterilisation inside packaging almost exclusively involve the use of sachets, which remain loose in the interior of the package after disinfection has occurred. Other disclosures involve disinfecting packaging which has the active agents incorporated into the packaging process via a metering nozzle which sprays or coats the active agents directly into the package before sealing, a system which carries a significant risk of exposure to operators. In other disclosures, the sterilising agent is added after sealing of the package via a needle or other means, compromising the integrity of the package post-sealing.

Other sterilisation technologies currently used include heat, chemicals such as ethylene oxide, irradiation (x-ray, gamma ray, electron beam), high pressure and filtration. All mentioned methods most often require dedicated facilities and personnel at an external site, increasing final product lead times and reducing cost effectiveness.

Ethylene oxide has been the choice of medical device packaging companies for the sterilisation of medical equipment in sealed plastic packaging. However, in 2016 the Environmental Protection Agency determined that ethylene oxide is significantly more dangerous than had previously been thought. Their report found that emissions from the Sterigenics medical sterilising plant in Willowbrook, Illinois could be harmful to public health. This report led the EPA to shut down the Sterigenics plant in February 2017 (Bloomberg Law, 2019). Further air quality testing in 2016 by the EPA concluded that ethylene oxide is thirty times more carcinogenic than was originally understood. Long term exposure can cause eye, lung, brain and nervous system damage. It has been shown to cause lymphomas, leukaemia, breast and other cancers. This information has led to the passing of tougher laws surrounding the use of ethylene oxide (Bloomberg Law, 2019). There is also evidence of an increased rate of miscarriage shown in female workers from inhalation exposure of ethylene oxide. It has also been shown that ethylene oxide is a mutagen which causes a decrease in testicular weight, sperm concentration, and testicular degeneration in animals with inhalation exposure (EPA, 2016).

Other logistical complications which can occur with the current use of ethylene oxide is movement of large batches of product to sterilising plants through different jurisdictions or legal areas, theft of shipments and the length of time taken to complete the shipping and sterilisation process. In 2019 the EPA closed three separate sterilising plants in the United States as a result of concerns over the carcinogenic effects of ethylene oxide to the surrounding community (Bloomberg Law, 2019).

There is therefore a need for a packaging system which facilitates decontamination and/or sterilisation of the packaged item(s) without requiring dedicated equipment or personnel at an external sterilisation facility or the like.

### SUMMARY OF THE INVENTION

According to the present invention there is provided a packaging system comprising a rigid container defining a main compartment for receiving an item to be packaged, the main compartment having a sealable opening for admission of the item into the main compartment; a module locatable in the container and operable through external activation to release a gaseous sterilising agent subsequent to sealing the opening; the container further defining an auxiliary compartment formed integrally with the main compartment and arranged to receive and retain the module in a fixed orientation such that the gaseous sterilising agent is directed into the main compartment.

Preferably, the container comprises a thermoformed plastic enclosure.

Preferably, the container comprises a blister pack.

Preferably, the auxiliary compartment is externally deformable to effect operation of the module to release the gaseous sterilising agent.

Preferably, the module is configured to be electromagnetically activated.

Preferably, the module is configured to be thermally activated.

Preferably, the module comprises first and second precursors of the sterilising agent, the module being arranged through external activation to bring the precursors into communication such as to generate the sterilising agent.

Preferably, the module comprises a first chamber containing the first precursor of the sterilising agent, a second chamber containing the second precursor of the sterilising agent, and a rupturable membrane separating the first and second chambers.

Preferably, at least a portion of a sidewall of the module is vapour permeable.

Preferably, the module comprises a first section and a second section secured together and between which the first element and the second element are contained, the first section and the second section being displaceable relative to one another from a first state into a second state such as to compress the module and rupture the membrane.

Preferably, the module comprises a reservoir of the first precursor in liquid form, the reservoir sealed by the rupturable membrane, and a substrate carrying the second precursor in solid form and positioned within the module to be in fluid communication with the first precursor when draining from the reservoir.

Preferably, the module defines a well in fluid communication with first precursor for receiving unreacted first precursor.

Preferably, the module comprises a key and the auxiliary compartment comprises a corresponding keyway arranged to receive the key only when the module in a specific orientation relative to the auxiliary compartment.

Preferably, the module comprises a plunger displaceable in response to an external force into rupturing engagement with the membrane.

Preferably, the plunger is displaceable in response to an external electromagnetic force.

Preferably, the plunger is displaceable linearly in response to undergoing rotary motion induced by the external force.

Preferably, the module comprises at least one deformable wall displacement of which brings the first and second precursors into communication.

Preferably, the module comprises an opposed pair of deformable walls.

Preferably, the packaging system comprises an externally visible indicator operable to provide a visual indication that the item has been sterilised.

Preferably, the visible indicator comprises a colour changing element.

Preferably, the first precursor comprises an acid and the second precursor comprises a salt.

Preferably, the first and second precursors are precursors of chlorine dioxide gas.

Preferably, the module is formed integrally with the auxiliary compartment.

According to a further aspect of the present invention there is provided a method of sterilising an item comprising the steps of providing a rigid container defining a main compartment for receiving the item and an auxiliary compartment integrally formed with the main compartment for receiving a module; locating the item in the main compartment; locating and maintaining the module in a fixed orientation in the auxiliary compartment; sealing the container; and externally activating the module to release a gaseous sterilising agent into the main compartment to sterilise the item.

Preferably, the method comprises the step of activating the module by externally deforming the auxiliary compartment.

Preferably, the method comprises the step of electromagnetically activating the module.

Preferably, the method comprises the step of thermally activating the module.

Preferably, the method comprises bringing first and second precursors into communication within the module in order to generate the gaseous sterilising agent.

Preferably, the method comprises rupturing a membrane within the module in order to bring the first and second precursors into communication.

Preferably, the method comprises the step of displacing a plunger through the membrane in response to an external force.

Preferably, the method comprises the step of visually indicating that the item has been exposed to the sterilising agent.

Preferably, the method comprises the step of thermoforming the rigid container.

Preferably, the method comprises the step of locating a key on the module into a keyway in the auxiliary compartment in order to specifically orient the module within the auxiliary compartment.

Preferably, the method comprises the step of effecting the relative displacement of first and second sections of the module in order to rupture a reservoir containing a precursor of the gaseous sterilising agent.

As used herein, the term “rigid container” is intended to mean a container defined by self-supporting walls such that the container is capable of retaining a fixed shape and internal volume in the absence of external forces but which may be flexible or otherwise deformable on the application of external pressure.

As used herein, the terms “decontaminate” and “sterilise” are used interchangeably and one should be considered to refer to and/or encompass the other.

### BRIEF DESCRIPTION OF THE DRAWINGS

The present invention will now be described with reference to the accompanying drawings, in which:

FIG. 1 illustrates a perspective view from above of a packaging system with integrated sterilisation according to an embodiment of the present invention, with a module positioned to be located in a container of the system;

FIG. 2 illustrates the packaging system of FIG. 1 with the module inserted into the container;

FIG. 3 illustrates a sectioned side elevation of the arrangement shown in FIG. 1;

FIG. 4 illustrates a sectioned side elevation of the arrangement shown in FIG. 2;

FIG. 5 illustrates the packaging system as shown in FIG. 2 with an external actuator being employed to effect actuation of the module;

FIG. 6 illustrates a perspective view from above of an alternative embodiment of a packaging system according to the present invention, showing a module positioned to be located in a container of the system;

FIG. 7 illustrates the packaging system of FIG. 6 with the module inserted into the container;

FIG. 8 illustrates a sectioned side elevation of the arrangement shown in FIG. 6;

FIG. 9 illustrates a sectioned side elevation of the arrangement shown in FIG. 7;

FIG. 10 illustrates the packaging system of FIGS. 6 to 9 having a closure secured to the container to seal the contents (not shown) thereof;

FIG. 11 illustrates a perspective view from above of a further alternative embodiment of a packaging system according to the present invention with a module positioned to be located in a container of the packaging system;

FIG. 12 illustrates the packaging system of FIG. 11 with the module inserted into the container;

FIG. 13 illustrates a sectioned side elevation of the arrangement shown in FIG. 11;

FIG. 14 illustrates a sectioned side elevation of the arrangement shown in FIG. 12;

FIG. 15 illustrates a sectioned front elevation of the arrangement shown in FIGS. 11 and 13;

FIG. 16 illustrates a sectioned front elevation of the arrangement shown in FIGS. 12 and 14;

FIG. 17 illustrates the arrangement shown in FIGS. 12 and 14 with the module compressed into an activated state;

FIG. 18 illustrates the packaging system of FIGS. 11 to 17 with a gaseous sterilising agent issuing from the module into a main compartment of the container of the packaging system;

FIG. 19 illustrates two separated halves of the module according to the embodiment of FIGS. 11 to 18 the packaging system of the present invention;

FIG. 20 illustrates the halves of the module shown in FIGS. 11 to 19 connected together in a first or expanded configuration; and

FIG. 21 illustrates the halves of the module shown in FIGS. 11 to 20 connected together in a second or compressed configuration.

### DETAILED DESCRIPTION OF THE INVENTION

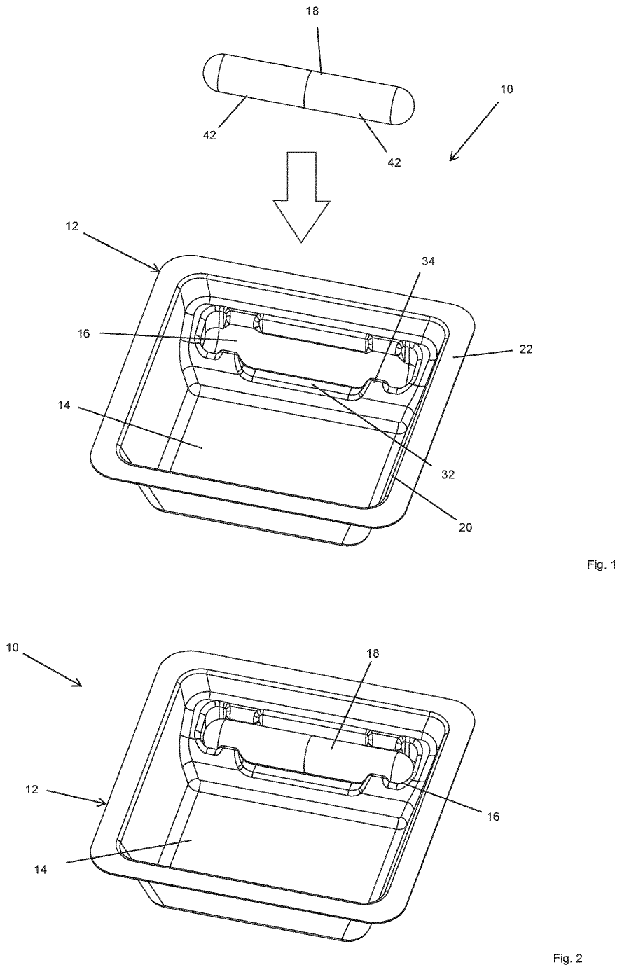

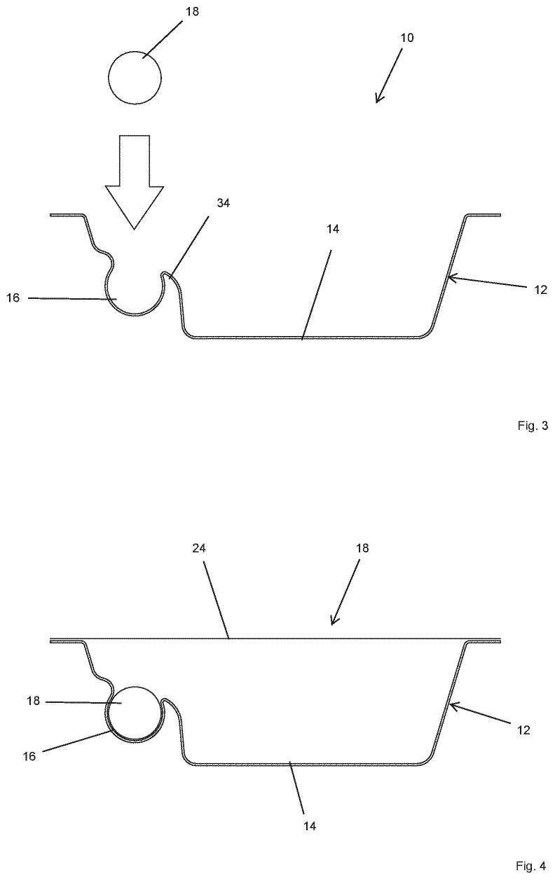

Referring now to FIGS. 1 to 5 of the accompanying drawings there is illustrated a packaging system with integrated sterilisation according to an exemplary embodiment of the present invention, generally indicated as 10. The packaging system 10 comprises a rigid container 12 for housing an item (not shown) to be packaged, for example a medical device, pharmaceutical product, food product, or the like, and which requires sterilisation or decontamination prior to use. The container 12 defines a main compartment 14 for receiving and retaining the item, in addition to an auxiliary compartment 16 integrally formed with the main compartment 14 and which, in use, is in vapour communication with the main compartment 14 as described hereinafter in detail. The auxiliary compartment 16 is shaped and dimensioned to receive a module 18 which is externally operable, as described in detail hereinafter, to effect the generation of a sterilising gas (not shown) after the container 12 has been sealed, which sterilising gas can flow into the main compartment 14 in order to comprehensively contact and therefore sterilise the packaged item contained therein.

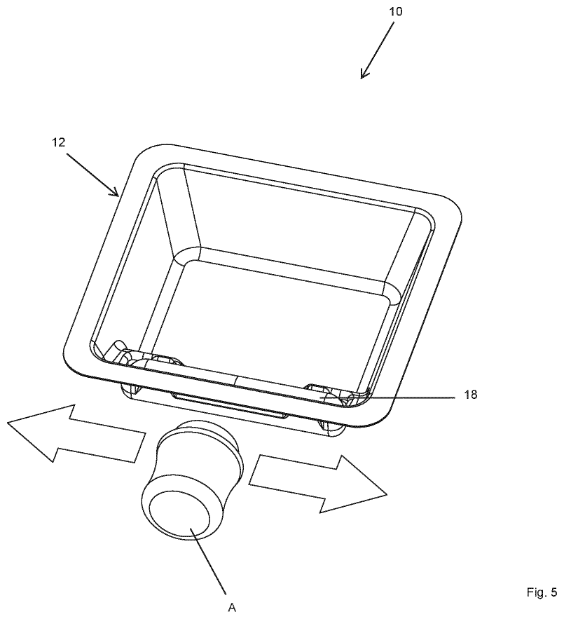

In this embodiment the module is cylindrical in shape, with the auxiliary compartment 16 having a complementary shape in order to securely receive the module 18 therein. It will of course be understood that any other suitable shape and size of module 18 and auxiliary compartment 16 may be employed. A depression defining an opening or window 32 is preferably provided or formed integrally in a retaining wall 34 formed between the main compartment 14 and the auxiliary compartment 16 in order to allow the sterilising gas to be dispensed into the main compartment 14. The container 12 defines an opening 20 via which an item to be packaged can be both introduced and removed from the main compartment 14. The container 12 may be sealed by any suitable means, for example the application of a film 24 (shown in FIG. 4) or more rigid closures (not shown) across the opening 20. The opening 20 may be defined by a mouth or rim 22 which may provide a surface to which said film or covering may be adhered, heat or otherwise sealed. It will of course be understood that the container 12 and the opening 20 may be of any other suitable shape and configuration, and the relative locations and dimensions of the main compartment 14 and auxiliary compartment 16 may be varied as required.

In order to produce the sterilising gas the module 18 preferably contains separate precursors of the sterilising gas, for example a first precursor located and retained in a first element such as one section 42 of the module, and a second precursor located and retained in a second element such as an other section 42 of the module 18 and which precursors may be brought into communication with one another by various mechanisms, in order to react and generate the sterilising gas. The module 18 is preferably provided with at least a section of gas permeable sidewall in order to allow the generated sterilising gas to issue from the module 18 to effect sterilising of the packaged item. For example the module 18 may include one or more openings or vents (not shown) in the cylindrical sidewall in order to facilitate the escape of the gaseous sterilising gas once the first and second precursors have been brought into contact. If said openings or vents (not shown) do not extend around the full circumference of the module 18 the packaging system 10 may include suitable features to ensure that the module 18 can only be positioned within the auxiliary compartment 16 such that the openings or vents (not shown) face into the main compartment 14. For example a key (not shown) may be provided on the exterior of the module 18 and a corresponding keyway (not shown) may be provided in the sidewall of the auxiliary compartment 16.

In the embodiment illustrated in FIGS. 1 to 4 the module 18 may be physically deformable in order to bring the separate precursors into contact, for example by means of external compression of the module 18 from the exterior of the container 12. However any other suitable means may be employed, for example as described hereinafter with reference to FIG. 5. For example the module 18 may comprise a rupturable reservoir (not shown) of a first precursor, with a second precursor located within the module 18 such as to be contacted by the first precursor once the reservoir has been ruptured, for example by compressing the module 18 to burst or otherwise open the reservoir of the first precursor. The first and second precursors are preferably provided in an amount which will generate a sufficient quantity of the sterilising gas to effectively sterilise the packaged item without excess. The module 18 is shown as an elongate cylinder or capsule form, but it will be understood that any other suitable form factor may be employed once embodying the above functionality.

As an exemplary alternative method of activation, and referring to FIG. 5, the module 18 may further comprise a rupturing element such as a plunger (not shown) positioned longitudinally within the module 18, at least a portion of the plunger comprising a magnetic material. Thus by the external application of a magnetic field, for example manually by means of an external actuator A, the plunger (not shown) can be caused to undergo linear displacement in order to contact and rupture the membrane (not shown). Any suitable mechanism may be employed to apply said magnetic force in place of the manually operable actuator A, and for example a packaging machine (not shown) which forms the container 12 may be arranged to apply the magnetic force at the appropriate point in the packaging process, most notably after the container 12 has been sealed with the item therein. Likewise the packing machine may be arranged to apply a physical force to the module 18 from the exterior of the container 12 once sealed, in the case where the module 18 is configured to be physically compressed to effect actuation.

The container 12 may be formed of any suitable material and by any suitable manufacturing means. In a preferred embodiment the container 12 is substantially or predominantly rigid and thermoformed from a suitable polymer or polymer blend, although suitable cold forming techniques may be employed. The main compartment 14 and auxiliary compartment 16 may be separated from one another by a gas permeable membrane (not shown) or the like, such that when the container 12 is opened by a user in order to retrieve the packaged item, the module 18 is not visible and/or accessible. The container 12 is formed as a single component such that the main compartment 14 and the auxiliary compartment 16 are integrally formed with one another. While the main compartment 14 is relatively rigid, for example in order to provide a suitable level of protection to the packaged item during storage and preferably to ensure the container 12 is self-supporting, the auxiliary compartment 16 or at least a portion thereof may be deformable, although this is not essential as will be apparent from the following description of the invention. The auxiliary compartment 16, or at least the deformable portion thereof, is accessible from an exterior of the packaging system 10 in order to allow a user or machine to grasp or otherwise engage the exterior of the auxiliary compartment 16, or to manipulate the module 18 therein, for example to compress or otherwise deform the auxiliary compartment 16. Thus when the packaging system 10 is sealed with an item in the main compartment 14 and the module 18 in the auxiliary compartment 16, an operative at the packaging facility, an item of machinery such as the packaging machine or downstream machinery, or an end user, can externally manipulate or compress the module 18 through the walls of the auxiliary compartment 16. The operative or machine can therefore initiate the generation of the sterilising gas which will then issue from the module 18 to sterilise the packaged item.

The use of a rigid container 12 in which the main compartment 14 and the auxiliary compartment 16 are formed integrally with one another ensure that the relative positions of the compartments 14, 16 are fixed, thus ensuring that the module 18 is retained in the correct position and orientation to achieve an effective discharge of the gaseous sterilising agent into the main compartment 14 and thus to reliably achieve the desired levels of sterilisation. The auxiliary compartment 16 is also configured to securely retain the module 18 therein, preferably in a fixed orientation as hereinbefore described, to again ensure reliable actuation and dispensing of the sterilising agent into the main compartment 14. In the embodiment illustrated this is achieved by dimensioning the inner sidewall of the auxiliary compartment 16 to have a greater than semi-circular length, as defined by the retaining wall 34, which thus requires the module 18 to be pressed into the auxiliary compartment 16 where the retaining wall 34 will then hold it in place. The walls of the auxiliary compartment 16 are therefore preferably sufficiently flexible, while being generally rigid, to allow resilient outward deformation to permit the module 18 to be pressed into position therein. It will of course be understood that alternative or additional mechanical features may be employed to securely retain the module 18 within the auxiliary compartment 16.

Furthermore, the use of a rigid contain 12 facilitates securing the item to be packaged (not shown) within the main compartment 14 in a fixed orientation, for example by employing one or more upstands or tabs (not shown) or the like, preferably formed integrally with the main compartment 14 during moulding, which tabs (not shown) can engage and retain the item (not shown) in a desired position within the main compartment 14. This may for example allow the item (not shown) to be raised off the base of the main compartment, and held in position so as not to be in contact with the sidewalls or closure 24. In this way the sterilising gas is allowed to contact the entire surface of the item, further ensuring reliable sterilisation.

While the module 18 is optionally deformable from an exterior of the container 12, it is also envisaged that a different arrangement may be employed in order to allow an alternative external force to initiate the generation of the sterilising gas. For example the module 18 could be internally separated into a pair of chambers (not shown) separated from one another by a frangible or rupturable membrane (not shown) or the like, each chamber containing one of the precursors of the sterilising gas. An actuator such as a plunger (not shown) could be located in one chamber, and could be displaceable by an external force into the membrane in order to allow the two precursors to come into contact. The plunger could for example be magnetic and displaced using a magnetic element drawn along the appropriate portion on the exterior of the auxiliary compartment 16 in order to force the plunger into the membrane, as described above in relation to FIG. 5. In addition the module 18 may be configured to be activated via other contactless means such as the use of electromagnetic or thermal activation or any other suitable wireless means including for example radiofrequency activation such as RFID, Bluetooth® Wi-Fi®, etc.

Although various sterilising gases may be employed with the packaging system of the invention, the use of chlorine dioxide (ClO₂) as the sterilisation gas or agent is particularly preferred. In the gaseous form chlorine dioxide does not require large and expensive equipment and specialised operators. Presently, commercial applications of ClO₂, include water disinfection, odour elimination and reduction of microbial organisms, with ClO₂ being dissolved in aqueous medium. Approved by the U.S. Food and Drug Administration (FDA) and the U.S. Environmental Protection Agency (EPA), chlorine dioxide is as powerful as peracetic acid but more economical, yet has far less of an impact on the environment than quaternary ammonium salts, chlorine or bromine. It costs about the same to use as other sterilisers or sanitizers but is more versatile and less harmful. It has also been shown to destroy and prevent biofilms, one of the most significant challenges to food processors in destroying harmful bacteria. It also does not have the strong odour or corrosive qualities associated with chlorine.

Chlorine dioxide (ClO₂) must be generated on site at the point of use. Although it is approved as a sterilant by the FDA, it is illegal to store or transport due to its volatile nature. Chlorine dioxide requires a dedicated facility and equipment, taking up space and adding significant expense as well as implementation of extensive safety measures such as gas containment and facility monitoring. Chlorine dioxide must; therefore, be generated in-situ. The packaging system 10 of the present invention is adapted to utilise in-situ chlorine dioxide generation on-site at point of use in a packaging facility or the like.

In the embodiments disclosed herein sodium chlorite and citric acid may comprise the precursors that are combined, for example within the module 18 to create the chlorine dioxide gas. It will however be appreciated that other acids and salts may be used to create the gas and the invention is not limited to this combination. The quantity and therefore concentration of chlorine dioxide can be accurately determined for a selected size of container 12. Obviously the concentration of sterilising gas can be adjusted through various means, most notably the quantity of precursors, to suit particular applications.

The packaging system 10 of the invention thus has the advantage of integrated self-sterilisation which may be performed easily by any user in any setting with minimal training. The time of sterilisation may be easily calculated for availability of contents, in particular the item(s) to be packaged. When chlorine dioxide is selected as the sterilising agent the by-products of the self-sterilisation are non-toxic, and disperse within the container 12 within a short time compared to the time required for aeration of products sterilised using ethylene oxide for example. In addition the by-products of the chlorine dioxide sterilant are non-toxic to the environment, unlike ethylene oxide.

Furthermore, items to be sterilised within the packaging system 10 can be sterilised over a relatively short period of time compared to the number of days required between conventional packaging and shipping sealed items such as medical devices to specialised sterilisation plants, sterilising for over twenty four hours and shipping the products back to the company prior to shipping to the consumer.

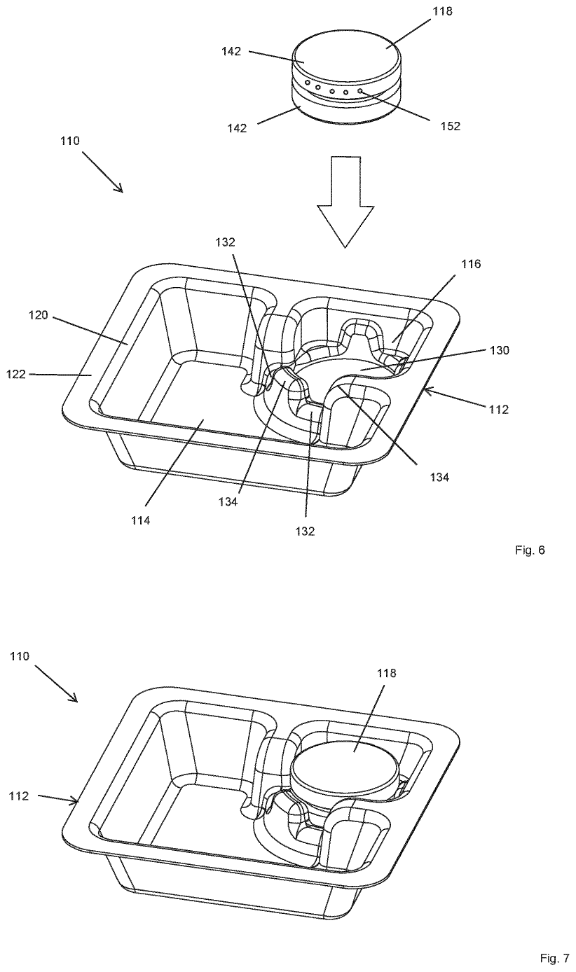

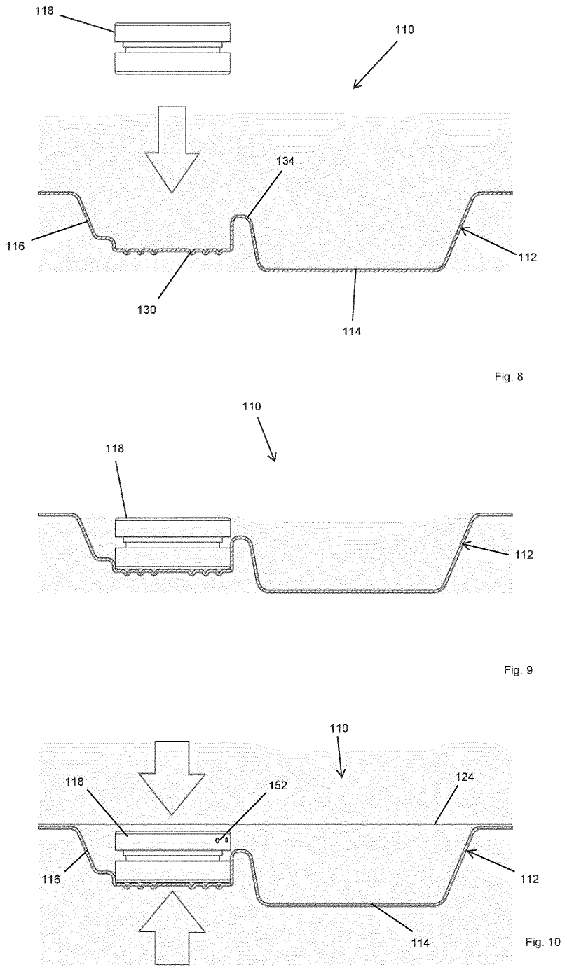

Referring now to FIGS. 6 to 10 there is illustrated an alternative embodiment of a packaging system according to the present invention, generally indicated as 110. In this alternative embodiment like components have been accorded like numerals, and unless otherwise stated perform a like function.

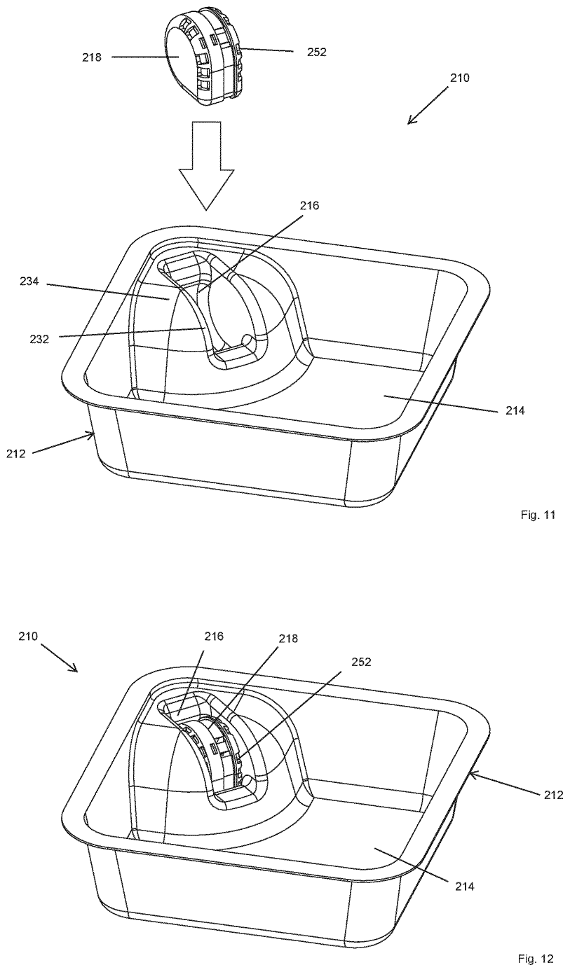

The packaging system 110 comprises a rigid container 112 defining a main compartment 114, an integrally formed auxiliary compartment 116 for receiving a module 118, and a sealable opening 120 providing access to the interior of the container 112. The container 112 is again preferably thermoformed with the main compartment 114 being sufficient rigid to provide the necessary level of protection and/or support to the item(s) to be packaged. The auxiliary compartment 116 may also be rigid, but includes at least one deformable wall, and in the embodiment illustrated a deformable base 130 which can be depressed upward from the underside as accessed from an exterior of the container 112. The deformable base 130 may for example comprise a number of concentric corrugations to allow the base 130 to flex or deform inwardly towards the module 118. In this way the module 118 may be compressed between a closure 124 sealing the container 12 and the deformable base 130. The module 118 is in the form of a short cylinder which is at least partially deformable in order to allow precursors contained therein to be physically brought into contact, for example as hereinbefore described, to generate a sterilising gas which can escape though a gas permeable portion of the module 118, for example vents 152 as shown in FIGS. 6 and 10. Thus in a particularly preferred arrangement first and second sections 142 of the module 118 are displaceable relative to one another in order to effectively compress the module 118, which in use is positioned directly against or adjacent the base 130 of the auxiliary compartment 116 and can therefore be manipulated by pressing inwardly against the base 130 and the closure 124 from the exterior of the container 112. A pair of openings or windows 132 formed integrally in a retaining wall 134 defining the auxiliary compartment 116 permits the passage of the sterilising gas into the main compartment 114 in use, as described above with respect to the previous embodiment. Again as with that embodiment the auxiliary compartment 116 is arranged to securely retain the module 118 therein in order to ensure that the module 118 remains in a fixed position and/or orientation relative to the main compartment 114. This is preferably achieved by way of an interference or overlapping fit between the module 118 and the auxiliary compartment 116.

With this exemplary construction of the module 118 the pair of sections or halves 142 have a first precursor 144 and a second precursor 146 captured therebetween. The first precursor (not shown) may for example be a rupturable pouch containing a liquid form of the precursor, while the second precursor (not shown) may be of solid form, for example the requisite chemical or compound in polymer form or one or impregnated in a suitable substrate (not shown). The halves 142 are suitably secured together with the first and second precursors captured therebetween, and the module 118 located in the auxiliary compartment 116 before the container 112 is sealed. At the appropriate time the module 118 can be manipulated from the exterior through the base 130 and/or closure 124 in order to initiate the generation of the sterilising gas to sterilise the item(s) (not shown) packaged within the packaging system 110. FIG. 10 illustrates this process but without an item being located in the main compartment 114. It can be seen that the vents 152 are oriented to face through the window 132 in the dividing wall 134 to direct the sterilising gas into the main compartment 114. As with the previous embodiment the packaging system 110 may include suitable features to ensure that the module 118 can only be positioned within the auxiliary compartment 116 such that the openings or vents 152 face into the main compartment 114. For example a key (not shown) may be provided on the exterior of the module 118 and a corresponding keyway (not shown) may be provided in the sidewall of the auxiliary compartment 116.

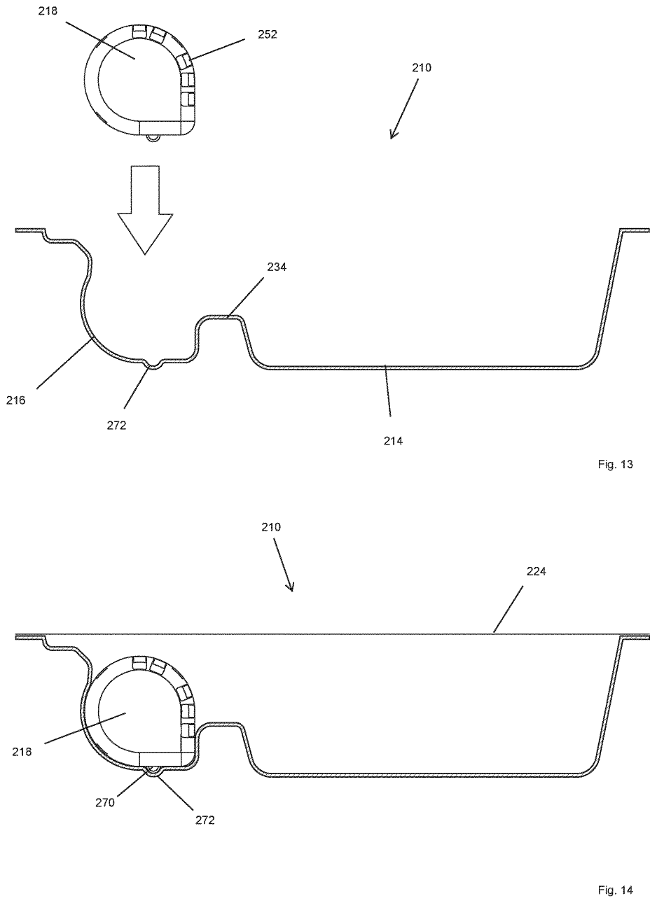

Turning to FIGS. 11 to 21 there is illustrated a further alternative embodiment of a packaging system according to the present invention, generally indicated as 210. In this alternative embodiment like components have been accorded like numerals, and unless otherwise stated perform a like function.

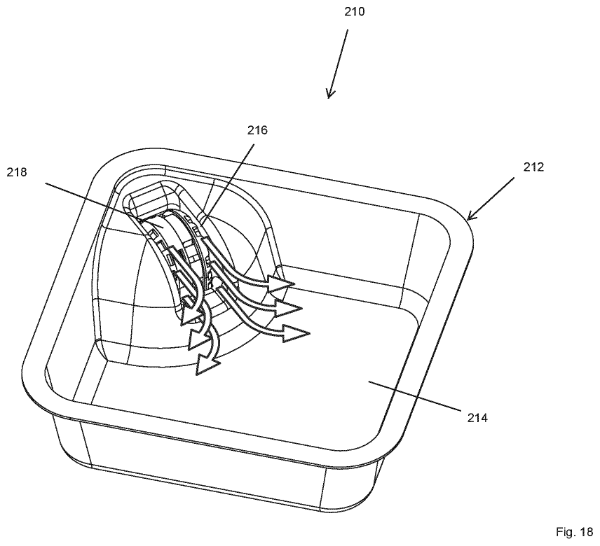

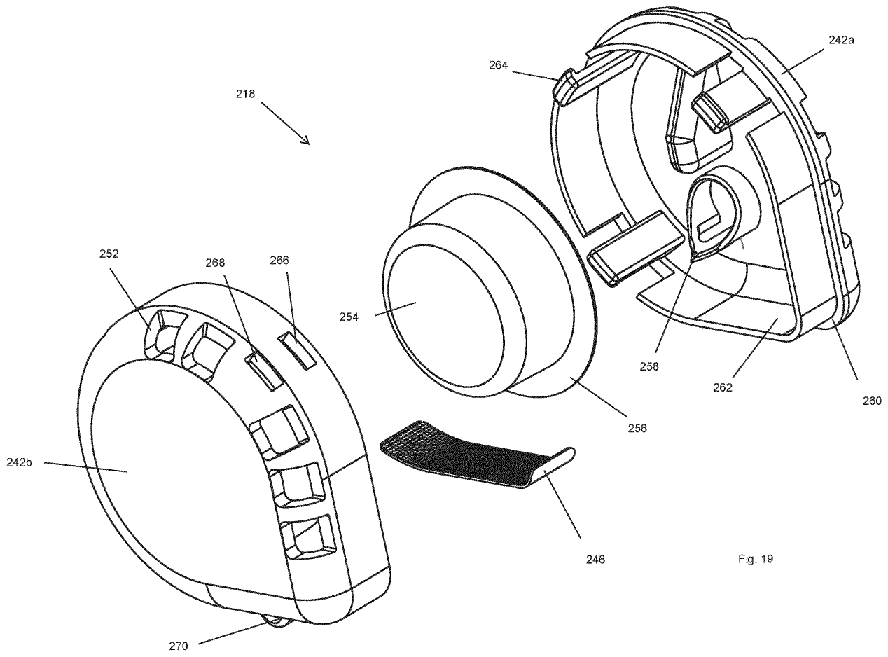

The packaging system 210 is similar in configuration to that of the embodiment of FIGS. 6 to 10, comprising a rigid and preferably thermoformed container 212 having a main compartment 214 and an integrally formed auxiliary compartment 216 for receiving and retaining a module 218 therein. The auxiliary compartment 216 is in the form of a slot or pocket defined in an integrally formed retaining wall 234 and having a pair of opposed walls 240 which are spaced from one another to accommodate the module 218. The auxiliary compartment 216 is again preferably dimensioned to generate an interference fit with the module 218, preferably circumferentially, in order to securely retain the module 218 in place. The module 218is again of a short substantially cylindrical form, but is received in a different orientation such that each of first section 242a and a second section 242b of the module 218 are adjacent one of the walls 240 of the auxiliary compartment 216. Both of the sections 242a, 242b are accessible from the exterior of the container 212 and are manually or machine deformable as hereinbefore described, in particular to displace the module 218 from a first state as shown for example in FIGS. 12 to 16 and 20, to a second relatively compressed state as illustrated in FIGS. 17 and 21. In this way both sections 242a, 242b of the module 218 may be indirectly engaged through the walls 240 to displace the module 218 from the first state into the second state, which as described hereinafter results in the generation of a gaseous sterilising agent within the module 218 and which is emitted from the module 218 into the main compartment 214. A portion of a sidewall of the module 218 may be at least in part gas permeable to allow the sterilising gas to escape from the module 218 and fill the main compartment 214 as illustrates in FIG. 18, for example by means of an array of vents 252.

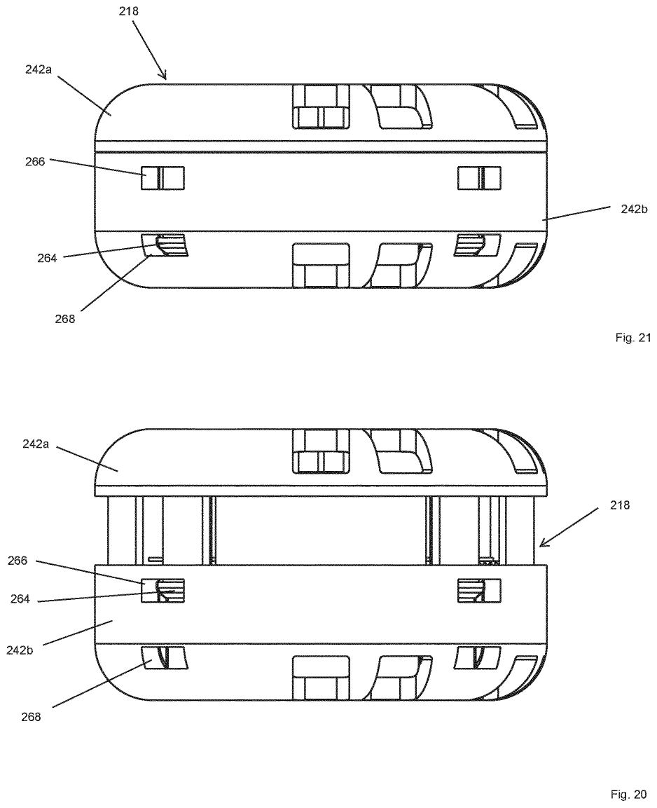

FIGS. 19 to 21 illustrate an exemplary construction for the module 218 which is similar in construction to the module 118. The module 218 again comprises the pair of sections halves 242a, 242b shown separated in FIG. 19, combined in the first state in FIG. 20 and in the second state in FIG. 21, and between which can be located a liquid first precursor (not shown) and a second precursor. The first precursor (not shown) is captured within a reservoir 254 sealed by a rupturable membrane 256, while the second precursor is impregnated in a mesh type substrate 246. The module 218 comprises a projection or plunger 258 extending from an inner wall of the first section 242a, and which is positioned adjacent the rupturable membrane 256 when the module is in the first state, and projects into and therefore pierces the membrane 256 when the module 218 is in the second state in order to release the liquid first precursor. The second precursor 246 is located to be in fluid communication with the liquid first precursor as it drains through the punctured membrane 256. The module 218 preferably defines a pointed or teardrop profile, extending at an in use lower end to a tip 260 which internally defines a well 262 into which can be captured any excess liquid first precursor that has not reacted with the second precursor, in order to avoid leakage of the liquid precursor from the module into the interior of the container 212. It will be understood that the relative positions of the reservoir 254, second precursor 246 and the well 262 may be varied while retaining the above described functionality.

The halves 242 are suitably secured together with the first and second precursors captured therebetween, and the module 218 located in the auxiliary compartment 216 before the container 212 is sealed by a closure 224 as hereinbefore described. In the exemplary embodiment illustrated the first section 242a comprises a plurality of barbed tabs 264 projecting towards and into register paired first sockets 266 and adjacent second sockets 268. This the first section 242a may be secured to the second section 242b in the first state by capturing the barbed tabs 264 in the corresponding first sockets 266. By pressing the first section 242a and second section 242b together the barbed tabs 264 will be pressed into the plurality of second sockets 268 in order to lock the module into the second state. This displacement into the second state also forces the projection 258 through the membrane 256 to initiate the reaction which generates the gaseous sterilising agent.

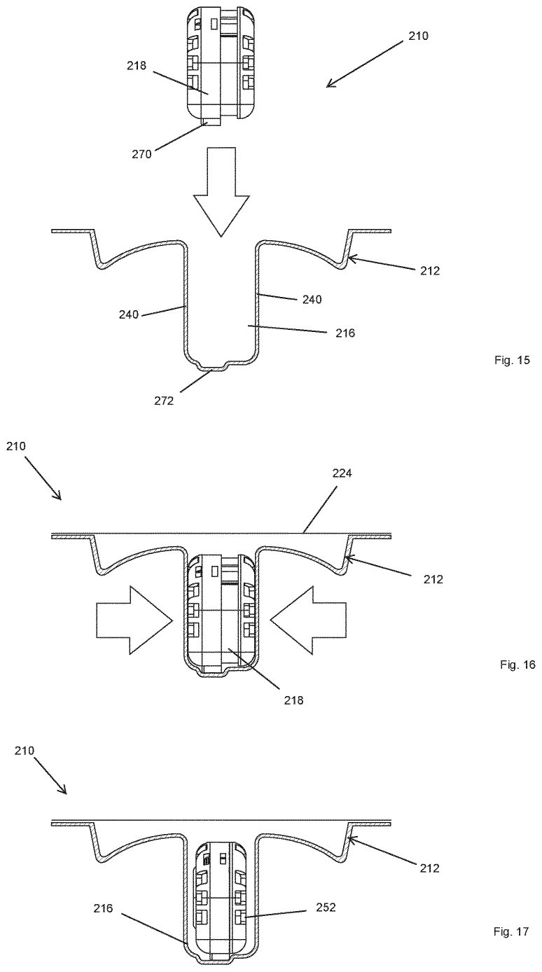

At the appropriate time the module 218 can be manipulated from the exterior through the walls 240 in order to initiate the generation of the sterilising gas to sterilise the item(s) (not shown) packaged within the packaging system 210. Such manipulation could for example be an automated procedure or step implemented as a stage of the packaging process. FIG. 16 includes a pair of arrows to illustrate this process, while FIG. 18 illustrates the module 218 within the container 212 but in the absence of the closure 224 and with sterilising gas being represented schematically issuing therefrom.

In order to achieve maximum exhausting of the sterilising gas into the main compartment 214 the openings or vents 252 do not extend around the full circumference of the module 218. Thus as detailed above the packaging system 210 may include suitable features to ensure that the module 218 can only be positioned within the auxiliary compartment 216 such that the openings or vents 252 face into the main compartment 214. For example a key 270 may be provided on the exterior of the module 218 and a corresponding keyway 272 may be formed integrally in the wall of the auxiliary compartment 216. The key 270 and keyway 272 are preferably positioned such that with the module 218 correctly and fully secured in the auxiliary compartment 216, and with the container 212 in a horizontal orientation such as resting on a horizontal surface, the tip 260 and thus the well 262 therein defines the lowest point of the module 218. In this way any excess liquid precursor will naturally drain into the well 262 for retention therein.

In any of the above embodiments the packaging system may be provided with an externally visible or accessible indicator (not shown) which is operable to indicate to an operator or end user that the sterilisation of the packaged item is complete. The indicator may for example take the form of a colour change indictor or the like.

The packaging system and method of present invention thus provides in-situ chlorine dioxide gas generation with on-site, at point of use and at-will convenience. This novel approach uses a unique delivery system with reactants contained in a discrete module or compartment which can either be removed from the package after sterilisation or hidden within the package due to its unique design, avoiding any unsightly materials left loose in the final packaging system post sterilisation.

The packaging system and method can be scaled to accommodate larger or smaller items to be packaged. The container can be fabricated by inexpensive means using existing technology already familiar to the packaging company, for example thermoforming and sealing.

The packaging system and method of the invention thus provides a relatively simply, safe, low cost and effective means of sterilising items within a sealed container.

1. A packaging system comprising a rigid container defining a main compartment for receiving an item to be packaged, the main compartment having a sealable opening for admission of the item into the main compartment; a module locatable in the container and operable through external activation to release a gaseous sterilising agent subsequent to sealing the opening; the container further defining an auxiliary compartment formed integrally with the main compartment and arranged to receive and retain the module in a fixed orientation such that the gaseous sterilising agent is directed into the main compartment.

2. The packaging system of claim 1 in which the container comprises a thermoformed plastic enclosure.

3. The packaging system of claim 1 in which the container comprises a blister pack

4. The packaging system of claim 1 in which the auxiliary compartment is externally deformable to effect operation of the module to release the gaseous sterilising agent.

5. The packaging system of claim 1 in which the module is configured to be electromagnetically and or thermally activated.

6. The packaging system of claim 1 in which the module comprises first and second precursors of the sterilising agent, the module being arranged through external activation to bring the precursors into communication such as to generate the sterilising agent.

7. The packaging system of claim 6 in which the module comprises a first element containing or comprising the first precursor of the sterilising agent, a second element containing or comprising the second precursor of the sterilising agent, and a rupturable membrane separating the first and second elements.

8. The packaging system of claim 6 in which at least a portion of a sidewall of the module is vapour permeable.

9. The packaging system of claim 7 in which the module comprises a first section and a second section secured together and between which the first element and the second elements are contained, the first section and the second section being displaceable relative to one another from a first state into a second state such as to compress the module and rupture the membrane.

10. The packaging system of claim 9 in which the module comprises a reservoir of the first precursor in liquid form, the reservoir sealed by the rupturable membrane, and a substrate carrying the second precursor in solid form and positioned within the housing to be in fluid communication with the first precursor when draining from the reservoir.

11. The packaging system of claim 10 in which the module defines a well in fluid communication with first precursor for receiving any unreacted first precursor.

12. The packaging system of claim 1 in which the module comprises a key and the auxiliary compartment comprises a corresponding keyway arranged to receive the key only when the module in a specific orientation relative to the auxiliary compartment

13. The packaging system of claim 6 in which the module comprises a plunger displaceable in response to an external force into rupturing engagement with the membrane.

14. The packaging system of claim 13 in which the plunger is displaceable in response to an external magnetic or electromagnetic force.

15. The packaging system of claim 14 in which the plunger is displaceable linearly in response to undergoing rotary motion induced by the external force.

16. The packaging system of claim 1 comprising an externally visible indicator operable to provide a visual indication that the item has been exposed to the sterilising agent.

17. The packaging system of claim 16 in which the visible indicator comprises a colour color changing element.

18. The packaging system of claim 6 in which the first precursor comprises an acid and the second precursor comprises a salt.

19. The packaging system of claim 6 in which the first and second precursors are precursors of chlorine dioxide gas.

20. The packaging system of claim 1 in which the module is formed integrally with the auxiliary compartment.

21. A method of sterilising an item comprising the steps of providing a rigid container defining a main compartment for receiving the item and an auxiliary compartment integrally formed with the main compartment for receiving a module; locating the item in the main compartment; locating and retaining the module in a fixed orientation in the auxiliary compartment; sealing the container; and externally activating the module to release a gaseous sterilising agent into the main compartment to sterilise the item.

22. A method according to claim 21 comprising the step of activating the module by externally deforming the auxiliary compartment.

23. A method according to claim 21 comprising the step of electromagnetically activating the module.

24. A method according to claim 21 comprising bringing first and second precursors into communication within the module in order to generate the gaseous sterilising agent.

25. A method according to claim 21 comprising rupturing a membrane within the module in order to bring the first and second precursors into communication.

26. A method according to claim 25 comprising the step of displacing a plunger through the membrane in response to an external force.

27. A method according to claim 21 comprising the step of visually indicating that the item has been exposed to the sterilising agent.

28. A method of claim 21 comprising the step of thermoforming the rigid container.

29. A method of any claim 21 comprising the step of locating a key on the module into a keyway in the auxiliary compartment in order to specifically orient the module within the auxiliary compartment.

30. A method of claim 21 comprising the step of effecting the relative displacement of first and second sections of the module in order to rupture a reservoir containing a precursor of the gaseous sterilising agent.
